(19) United States
(12) Patent Application Publication
(10) Pub. No.: US 2021/0088682 A1
(43) Pub. Date: Mar. 25, 2021

(54) Readout Board Muxing for PET Systems

(71) Applicant: Sino Canada Health Engineering Research Institute (Hefei) Ltd., Hefei (CN)

(72) Inventor: James Schellenberg, Winnipeg (CA)

(21) Appl. No.: 17/026,858

(22) Filed: Sep. 21, 2020

Related U.S. Application Data
(60) Provisional application No. 62/904,247, filed on Sep. 23, 2019.

(51) Int. Cl. G01T 1/29; G01T 1/161; A61B 6/03

(52) U.S. Cl. CPC G01T 1/2985; A61B 6/037; G01T 1/1615

(57) ABSTRACT

Described herein is multiplexing scintillation blocks, called interblock muxing. Specifically, the start of an annihilation event is recorded and assigned a time stamp while the energy of the entire event is recorded separately. All events occurring at a series of multiplexed scintillation blocks are reported to a processor which distinguishes individual events and assigns the start of each event with its corresponding energy, thereby allowing for cheaper and more efficient processing of events during PET imaging.

### PRIOR APPLICATION INFORMATION

The instant application claims the benefit of U.S. Provisional Patent Application 62/904,247, filed Sep. 23, 2019, entitled “Readout Board Interblock Muxing for PET Systems”, the entire contents of which are incorporated herein by reference for all purposes.

### BACKGROUND OF THE INVENTION

PET medical imaging systems are typically arranged with a multitude of scintillation elements and readout boards organized around the object to be imaged. A PET system coincidence occurs when two scintillations occur at the same time, which provides a line of response along which the annihilation event must have occurred. These annihilation events occur inside the item being imaged.

For scintillator elements arranged around a body, some coincidence geometries are impossible, such as coincidence pairs that define a line of response that does not go through the body being imaged.

Multiplexing is commonly discussed within PET readout methods. Multiplexing is a way of reducing the number of cables that come out of the scintillator block, and also leads to channel count reduction and cost reduction. This type of multiplexing refers to using resistive readout, capacitive readout, or hybrid readout methods with an array of pixels. This multiplexing occurs at the level of one block and can be called intra-block muxing. More unique methods to perform multiplexing have been discussed in U.S. Pat. No. 9,903,961 by Ng et al. In this approach multiplexing is applied to the row and column organization of the pixels. This is still a multiplexing at the intra-block level. Multiplexing of the fast outputs of the pixels is also known in the art and is used to reduce the number of signals that need to be processed.

### SUMMARY OF THE INVENTION

According to an aspect of the invention, there is provided a method for distinguishing one scintillation event from a plurality of scintillation events at a series of scintillation blocks that are multiplexed together comprising:

each respective scintillation block detecting a start of a respective one annihilation event as a respective one annihilation event fast output, said respective scintillation block reporting the respective one annihilation event fast output to a processor, said processor applying a time stamp to the respective one annihilation event fast output;

each respective scintillation block measuring energy of the respective one annihilation event as a respective one annihilation event slow output voltage signal and reporting the respective one annihilation event slow output voltage signal to the processor, said processor applying a time stamp the respective one annihilation event slow output voltage signal;

said processor comparing respective one annihilation event fast output time stamps and respective one annihilation event slow output voltage signal time stamps to assign a respective one fast output and a respective one slow output voltage signal to a scintillation event.

According to another aspect of the invention, there are provided two or more scintillation blocks multiplexed together in series, each scintillation block comprising a scintillation photomultiplier (SiPM) board having a plurality of SiPM pixels, each respective one SiPM pixel of the plurality of SiPM pixels arranged proximal to a respective one corner of the respective scintillation block, each SiPM pixel having a fast output and a slow output;

each fast output on a respective scintillation block being multiplexed together for reporting a scintillation event on the respective scintillation block;

each slow output at the respective one corner of a first scintillation block being multiplexed to a slow output at a corresponding corner of at least a second scintillation block for determining energy of a scintillation event and relative location on a scintillation block where the scintillation event occurred.

According to another aspect of the invention, there is provided a method for distinguishing one scintillation event from a plurality of scintillation events at a series of scintillation blocks that are multiplexed together comprising:

providing two or more scintillation blocks multiplexed together in series, each scintillation block comprising a scintillation photomultiplier (SiPM) board having a plurality of SiPM pixels, each respective one SiPM pixel of the plurality of SiPM pixels arranged proximal to a respective one corner of the respective scintillation block, each SiPM pixel having a fast output and a slow output;

each fast output on a respective scintillation block being multiplexed together for reporting a scintillation event on the respective scintillation block;

each slow output at the respective one corner of a first scintillation block being multiplexed to a slow output at a corresponding corner of at least a second scintillation block for determining energy of a scintillation event and relative location on a scintillation block where the scintillation event occurred;
detecting one scintillation event at the multiplexed fast outputs on a respective one scintillation block, said respective one scintillation block reporting the one scintillation event fast output to a processor, said processor recording the one scintillation event fast output and applying a time stamp to the one scintillation event fast output;
the one scintillation event being measured by the multiplexed slow outputs at each corner of the series of scintillation blocks, said slow outputs each reporting the respective one scintillation event slow output and the measurement of the respective one scintillation event slow output to the processor, said processor applying a time stamp to each of the respective one scintillation event slow outputs measurements;
said processor comparing scintillation event fast output time stamps and respective one scintillation event slow outputs and assigning a scintillation event fast output and scintillation event slow outputs to one scintillation event, thereby mapping the one scintillation event to a specific location on a specific scintillation block.

### BRIEF DESCRIPTION OF THE DRAWINGS

FIG. 1 is a schematic diagram of the detector module.

FIG. 2 is a schematic diagram of the Brain PET mechanical system design.

FIG. 3 is a diagram of the CCB board connection details.

FIG. 4 illustrates how Fast 1 occurs as a separate input.

FIG. 5 shows a, b, c and d outputs of a given block.

FIG. 6 is a circuit diagram.

FIG. 7 shows fast and slow outputs offset from each other for clarity.

FIG. 8 shows combined outputs of FIG. 7.

FIG. 9 shows the Fast 2 output from block 2.

FIG. 10 shows another scintillation event occurring in LYSO block 2.

FIG. 11 shows the combined event as seen by the interblock muxing circuit.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is another type of multiplexing which is between blocks, called interblock muxing. A block consists of the scintillation material, the SiPM board, and the associated gels and masks that are used to optimize the optical coupling between these elements. The block may also include a light guide and light shield depending on the details of the design, as is known in the art. The block may also contain resistors and/or capacitors to allow 4 corner readout methods to be used on the block. The block may also contain resistors and capacitors to allow the multiplexing of the fast pixel outputs. These methods are known in the art.

Specifically, if one arranges the blocks in such a way that multiple blocks are connected, and that a priori due to geometry it is not possible to have a coincidence between these blocks, then it is possible to save on data acquisition circuits using circuit muxing techniques. These interblock muxing techniques can allow for a savings of money for manufacturing the circuits.

Specifically, as a pair of annihilation photons move in approximately opposite directions, if a specific block registers one annihilation photon, only a finite number of opposing blocks can register the corresponding annihilation photon. In the examples discussed herein, it is to be understood that there is a corresponding event taking place at a radially opposed block. It is also important to note that in most embodiments, there will be multiple radially opposed blocks that form the PET ring.

As discussed and demonstrated herein, this interblock muxing causes special programming to be possible, which is to use the fast pulse signals to indicate both the timestamp of a scintillation event as well as the block in which the event has occurred. In addition, these fast signals can be used to assist in determining when overlapping scintillation events have occurred in blocks. This overlap of the scintillation events can be accurately modeled using known exponential decay curves for scintillation detectors, which means that multiple overlapping scintillation events can be successfully distinguished from the combined slow output voltage signals. This type of signal processing is a method of reducing the pileup effect in interblock muxing systems.

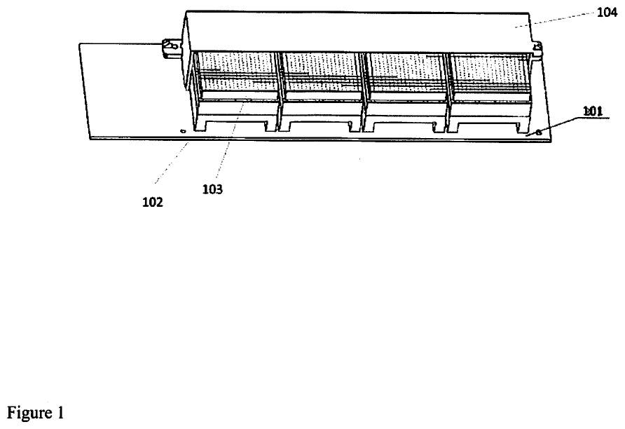

Assume a Brain Imaging PET system that is designed to work within an MRI bore. The PET system will have a readout board system as shown in FIG. 1, which shows 4 blocks made up of a scintillator board 103 and a SiPM board 102 which are attached to a Cassette Carrier Board (CCB) 101. Overtop of the four blocks is a scintillator cover 104 which keeps the blocks in optical darkness but which lets higher energy photons through. Specifically, FIG. 1 shows CCB Board 101 with 4 Blocks (102+103) and Scintillator Cover 104. The SiPM board 102 and Scintillator 103 are exposed. Optical Gel, masks and other details of the block are not shown in FIG. 1.

The SiPM board will have multiple SiPM pixels arranged in a grid, typically 4×4, 5×5, 6×8 etc. The slow output pins of these pixels are connected together using a resistor or capacitor grid, as is known in the art, and the output of the SiPM board is reduced to 4 slow corner outputs called a, b, c, and d. These corner output voltages can be used to determine the energy of a scintillation event and the x,y location on the scintillator block where the scintillation event occurred. Each SiPM pixel also has a fast output, and all of these fast outputs can be multiplexed together in a manner known in the art to allow a single fast output to exit the SiPM board. From each SiPM board there is therefore 4 slow outputs a, b, c, d and 1 fast output which become inputs to the CCB board.

There are 4 such SiPM boards in this example, and therefore without intra-block multiplexing the CCB board will receive 16 slow outputs (4 from each block) and 4 fast outputs (1 from each block). In this example, it is assumed that the slow and fast interblock multiplexing occurs on the SiPM board, but for PCB real estate reasons it is possible to perform interblock multiplexing on the CCB board instead. The same principles and methods apply, known in the art.

In order to achieve higher sensitivity, one must design more of the scintillator material around the area to be imaged. For this reason, there may be several of these CCB boards arranged around the field of view and so it is useful to have techniques which minimize the amount of cabling that is required. An additional purpose of the design is to reduce the amount of digitization circuits required for the design. It is an additional purpose to reduce the amount of heat generated and space required for the electronics, which is done by reducing the number of digitization circuits required.

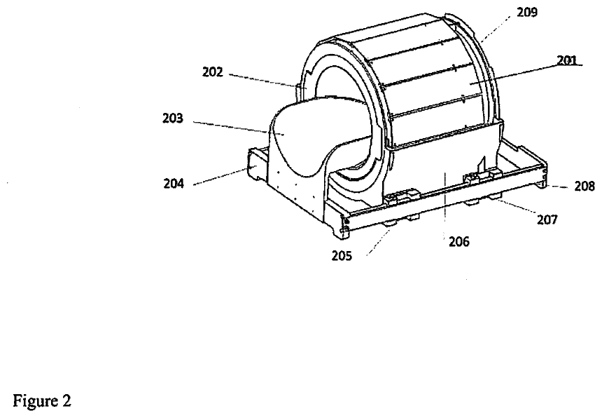

Assume that the PET System is cylindrical, and that the CCB board is arranged along the axial direction, and that one has a PET imaging device with 4 blocks per CCB along the axial direction and 16 CCB boards in the circumferential direction. This is shown in one exemplary embodiment in FIG. 2. Specifically, in FIG. 2, Tthe CCB boards 201 are arranged around the circumference of the PET Ring Inner Cylinder 209. Inside the PET Ring Inner Cylinder is mounted the MRI coil 202, which fits as a cylindrical coil inside the PET ring. The headrest 203 is attached to the front brace 204. The PET ring and MRI coil assembly (a combination of 202, 209, 206, 201) slides forward and back on the slide 208, which has front stop 205 and back stop 207 to control how far back and forward the assembly moves. The part 206 is the PET Ring Lower Cover. The PET Ring Upper Cover and associated cabling for MRI coil and PET Ring are not shown.

Assume that each block is readout using a mixture of slow and fast SensL pixel outputs. The fast and slow outputs are described by SensL documentation and are consistent with the use of 60035 or 30035 or 40035 J series pixels from SensL.

Assume one connects the fast outputs together for each block, in a manner that has been described by other authors. Assume that the slow outputs from the pixels are read out using 4 corner techniques, as described by other authors. As will be appreciated by one of skill in the art, the slow outputs can be used to calculate the specific location of the event in the block, and also to calculate the energy of the event. However, other geometries are possible and as such the invention is not necessarily limited to this specific orientation. For example, any suitable geometric shape, some of which may have less than or more than four corners, may be used within the invention.

The 4 blocks in the axial direction cannot be in coincidence with each other, and for low source strengths one can assume that there is only one event occurring at a time. Assume that the noise floor on the output A, B, C, D lines is quite low compared to the voltages that are read for an event. Noise on these output lines can come from LYSO radioactive noise, internal noise from the electronic circuits, or noise generated by the MRI system.

In this case, at these low source strengths, the entire set of 4 blocks can be readout by ganging the A, B, C, and D corner outputs together, and by using the fast output from each block to indicate which block is having an event. The fast outputs therefore become a block selector as well as a timing detector. The slow outputs continue to be used to calculate energy and x-y position.

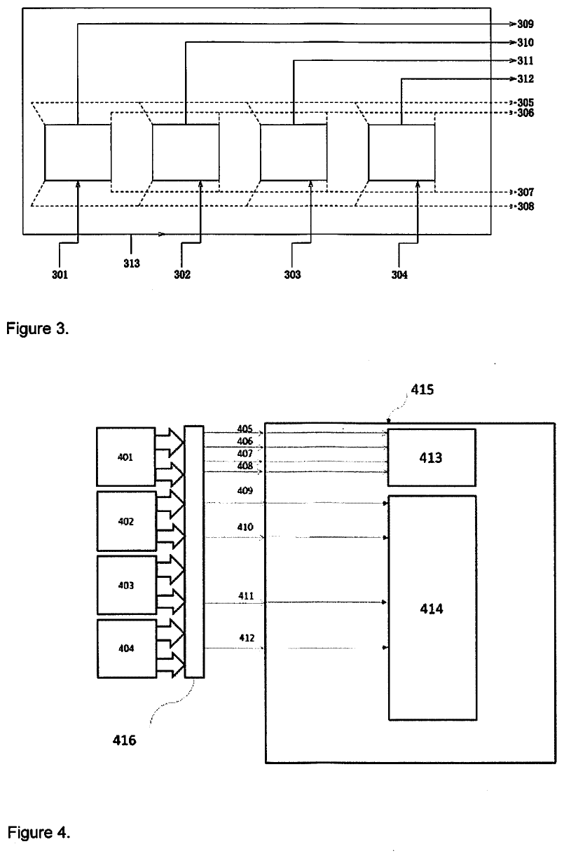

This CCB board design for an interblock muxing system is shown in FIG. 3. The CCB board 313 has four blocks connected on it, with Block 1 301, Block 2 302, Block 3 303 and Block 4 304 being spaced and located on the CCB in a manner suitable for the PET system design being done. For each block, there is an output A, B, C, D respectively from each corner of the block. In output line 305 all 4 lines A are connected together, A1, A2, A3 and A4 with the number designating which block the A line is from. Similarly, all 4 lines B are connected together to form output line 306, all C lines are connected together to form output 307 and output 308 is the ganging together of the D lines. Accordingly, corner A on block 1 corresponds to corner A on block 2 in that both corner As are in the same position relative to the overall geometry of the block. These lines are output from the CCB connector and terminate in a data acquisition system. In addition, there are 4 fast outputs, one for each block. Fast output from block 1 309, fast output from block 2 310, fast output from block 3 311 and fast output from block 4 312 are shown connecting the edge of the CCB board, and are then cabled off the board to the data acquisition system being used.

The approach at multiplexing outlined here will create 4 corner outputs connecting the respective and corresponding corners of all four blocks together and 1 fast output for each of the four blocks, for a total of 8 lines. This can be compared to standard readout methods where there are 4 corner outputs and 1 fast output per block, which would result in a total of 20 lines. This approach allows a cable size reduction from 20 lines to 8 lines, a reduction of 60%. This approach can be used with 2, 3, 4, 5 etc numbers of blocks, as long as the blocks are organized so that it is impossible for them to be in coincidence.

Each block of a scintillator detector system outputs 1 fast output and 4 slow outputs, labelled a, b, c, d. The fast output can be put into a TDC circuit for quick timestamping, and the slow outputs can be typically input to a 40 MHz ADC system to allow 25 nsec ADC samples to be taken. The fast output occurs quickly, with an approximate timescale of 1 nsec. for total duration. The TDC circuit commonly can be used to generate 25 psec. resolution or faster. The slow outputs occur slowly, due to the timescale of the photon decay in the scintillator and the due to the electronics timing delay related to RC time constants. Typical timescales for the slow outputs are 300 to 700 nsec. For example, the timescales for the Sensl SiPM pixels vary depending on the size of the SiPM that is used, with the 3 mm SiPM being fastest and the 6 mm being slowest. The slow outputs are used to determine the energy value of the event, and the fast output allows accurate timing of the event, as discussed below. One TDC and 4 ADC inputs are used to read these 5 block outputs.

This process is discussed in greater detail below with reference to FIGS. 4-11 for illustration.

Specifically, FIG. 4 shows the circuit connection from the CCB board 416 through to the data acquisition system 415. The data acquisition system 415 consists of a timing and block detection system 414 and an energy and x,y position calculation section 413. This data acquisition system 415 may consist of a high speed ADC system which connects via fiber to a workstation in the MR control room which provides x-y and energy calculation, or the data acquisition system 415 may use FPGA and other circuit techniques to perform x-y and energy calculation. The specific number of boxes or location of boxes does not alter the basic concept. On the CCB board 416 are blocks 401, 402, 403 and 404. These 4 blocks all connect to the CCB with slow and fast outputs as previously discussed. The slow and fast outputs are multiplexed down to a single set of slow outputs 405, 406, 407 and 408 and a set of 4 fast outputs 409, 410, 411, and 412.

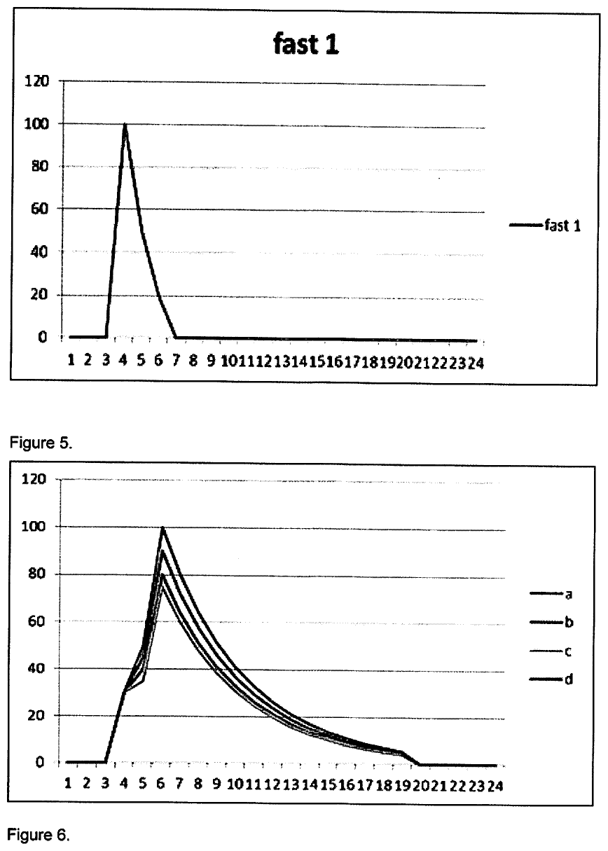

In FIG. 5 is shown a typical operation occurring in this system for the case where the radioactivity level is low. The Fast 1 signal is pulled high in a sharp manner. As will be apparent to those of skill in the art, this “fast 1” input is caused by a first scintillation event occurring in Block 1. The fast 2, fast 3 and fast 4 input lines did not pull to a sharp and high level and are not shown.

FIG. 6 shows the four slow outputs corresponding to this scintillation event, each measured at a corner of the given block, as discussed below, are exponential with a sharper front and a slower back. These “slow” outputs represent the summing of the event, as discussed below. These fast and slow outputs are assumed to be occurring from the same scintillation event, because the fast 2, fast 3 and fast 4 outputs have not had a sharp pulse occur.

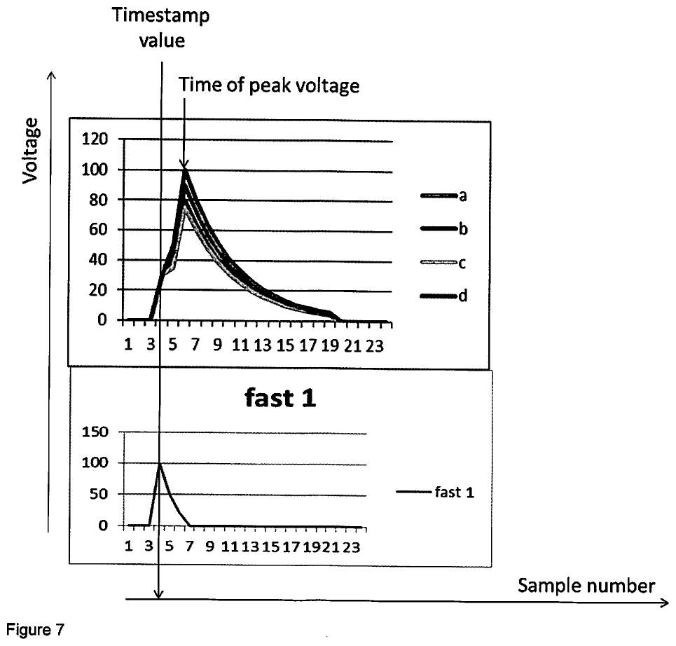

FIG. 7 shows the fast and slow outputs of the CCB on one timeline. Fast 2, Fast 3 and Fast 4 outputs are not shown because they did not pull sharp and high. The slow lines will typically be sampled at 40, 60 or 80 MHz, whereas the time input may be connected via TDC methods that are known in the art. There is a common time capability for the data acquisition system, which may be implemented in electronics or firmware or software, which allows for the fast and slow signals to be placed on a common time system.

It is clear that there is a timelag between the maximum voltage of the fast 1 output and the maximum voltages of the A, B, C and D slow lines. This time lag is due to the differences in RC time constant for these different systems, and due to the differences in the rate and method of sampling. The position of the slow signal maximum voltages and the relative height of the 4 slow signals will vary depending on the details for where on the block the scintillation event occurred. This variation in the height and time of the maximum values may also be modified by the temperature of the block and the bias voltage that is used with the pixels. For a given temperature, location and bias voltage, the relative heights and times are preserved constant across multiple scintillation events. This time lag value between fast and slot signals on each block can be used to assist in separating overlapping scintillation events on different blocks, as discussed below.

In FIG. 7, it is apparent that the time of peak voltage for the fast signal will in general be different than the time of peak voltage for slow A, which is again different from time of peak voltage for slow B, which is again different from peak voltage time of Slow C, and also of Slow D. These 5 different time values could be combined in various ways to create a timestamp. One method is to take the time of fast 1. Another might be to average over the 5 times. Another would be to weight the fast time more favourably than the slow times. Another would be to assume that the fast time is some portion of time after the actual event due to delays in the electronics and scintillator material. Regardless of the different methods of arriving at a timestamp, the interblock muxing techniques discussed here still apply.

In addition, there will be in general 4 different timelag values. One value between the peak voltage of the fast signal and peak voltage time of the slow A signal. Another timelag between fast and slow B. similarly another between Fast and Slow C and Fast and Slow D. There are various algorithms and methods that can be designed to calculate the single timelag value between the fast and slow signals. Regardless of the exact method that is used, the interblock muxing methods discussed here still apply.

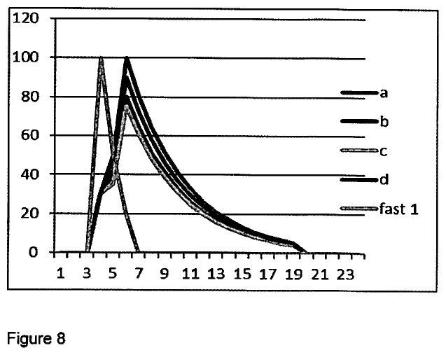

FIG. 8 shows the same curves from the scintillation event in FIG. 7, but on the same relative voltage and common timeline.

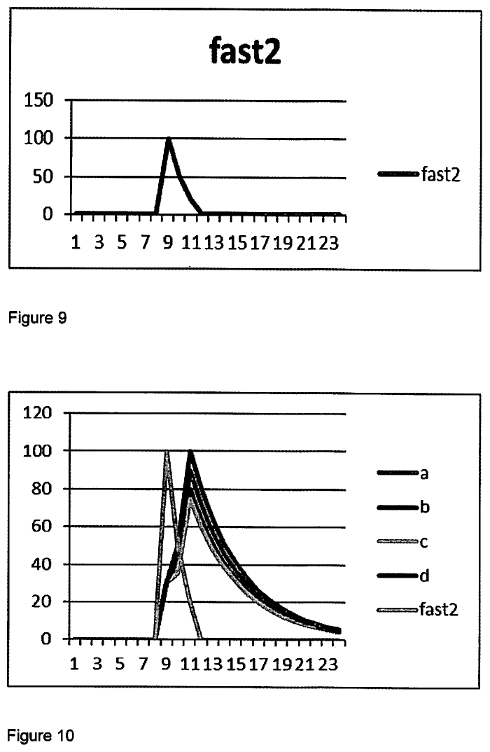

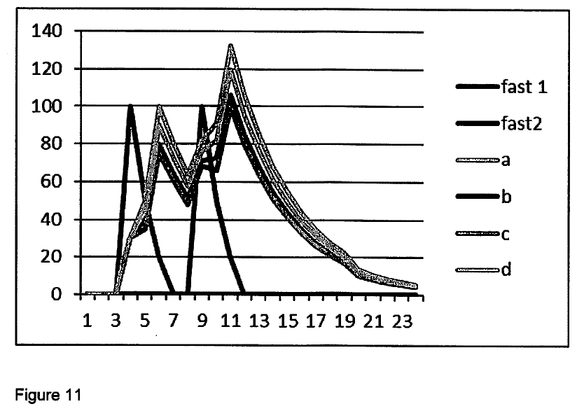

At low levels of radioactivity, it can be expected that the scintillation events on the CCB occur slowly and separated in time. As the radioactivity level of the object being imaged increases, there will start to be more than one scintillation event occurring on the CCB. For example, a scintillation event may occur in Block 1 and a separate scintillation event occurs in Block 2, 3 or 4 almost at the same time as each other. To illustrate this, FIG. 9 shows a “Fast 2” output from block 2 for reference purposes. FIG. 10 shows the voltage values that occur from A, B, C and D slow inputs. FIG. 11 shows the combined output of the CCB in the that the scintillation event on Block 1 occurs nearly at the same time as the scintillation even in Block 2.

As discussed above, blocks 1 and 2 are multiplexed and as such in practice these two events would be reported to the same circuit, as illustrated in FIG. 11. As can be seen, in this case, because outputs a, b, c, d of the various blocks are tied together the voltage outputs of block 1 and 2 are summed together and appear at the ADC system together. These 2 overlapping voltage curves need to be seperated in hardware, firmware and/or software if they are to be recognized as separate events. The Fast outputs fast 1 and fast 2 remain as separated inputs, and can be used to indicate to the signal processing software that 2 events have occurred. The signal processing software also knows the typical timelag that occurs in those blocks for a given combination of block temperature, bias voltage and x,y location. This allows the data acquisition system to deconstruct the 2 events, coming up with separate time stamps, energy and x-y location information for each event.

An additional advantage of this technique is that the number of analog to digital conversion systems that will be required is reduced by a factor of 4. For the CCB board below, instead of 16 ADC ports in the standard connection case we have 4 ADC ports. This leads also to a 75% reduction in heat load for the system and a 75% reduction in the board area required to implement the ADC circuits. In addition, there is a 75% reduction in circuit cost for these systems. In addition, it is reasonable to also expect a significant reduction in cooling costs and space, if cooling systems are required within the PET system. In addition, the reduction in required circuit size for the ADC circuits may allow shorter connection paths to be achieved between CCB board and ADC circuit, which is expected to allow improved performance of the PET system. This reduction in number of ADC system will be the same as the number of blocks that are multiplexed.

For all PET systems that are implemented within the MRI bore, the reduction in heating, space, cost, cooling and cable requirement may allow novel design approaches to be used. These novel design approaches include the implementation of the ADC circuits within the MRI bore. The ADC circuits in some cases may be design directly on the CCB board itself, depending on the size of the circuits.

According to an aspect of the invention, there is provided a method for distinguishing one scintillation event from a plurality of scintillation events at a series of scintillation blocks that are multiplexed together comprising:

each respective scintillation block detecting a start of a respective one annihilation event as a respective one annihilation event fast output, said respective scintillation block reporting the respective one annihilation event fast output to a processor, said processor applying a time stamp to the respective one annihilation event fast output;

each respective scintillation block measuring energy of the respective one annihilation event as a respective one annihilation event slow output voltage signal and reporting the respective one annihilation event slow output voltage signal to the processor, said processor applying a time stamp the respective one annihilation event slow output voltage signal;

said processor comparing respective one annihilation event fast output time stamps and respective one annihilation event slow output voltage signal time stamps to assign a respective one fast output and a respective one slow output voltage signal to a scintillation event.

In one aspect of the invention, there are provided two or more scintillation blocks multiplexed together in series, each scintillation block comprising a scintillation photomultiplier (SiPM) board having a plurality of SiPM pixels, each respective one SiPM pixel of the plurality of SiPM pixels arranged proximal to a respective one corner of the respective scintillation block, each SiPM pixel having a fast output and a slow output;
each fast output on a respective scintillation block being multiplexed together for reporting a scintillation event on the respective scintillation block;
each slow output at the respective one corner of a first scintillation block being multiplexed to a slow output at a corresponding corner of at least a second scintillation block for determining energy of a scintillation event and relative location on a scintillation block where the scintillation event occurred.

In some embodiments, the scintillation blocks according to claim 1 wherein the scintillation blocks are multiplexed to a Collimator Control Board.

There are more than 2 scintillation blocks multiplexed in series. For example, there may be 3, 4, 5, 6 or more multiplexed together.

Each scintillation block may have more than 3 corners. Specifically, in the examples discussed herein, each scintillation block has 4 corners. However, other suitable geometric shapes having more or less corners may be used within the invention, as discussed herein.

In some embodiments of the invention, there are 4 scintillation blocks multiplexed in series, each scintillation block having 4 corners.

In some embodiments, the scintillation blocks further comprise a third scintillation block and a fourth scintillation block, said scintillation blocks being arranged axially, each scintillation block having an upper right corner, an upper left corner, a lower right corner and a lower left corner, each slow output at the upper right corner of each scintillation block being multiplexed together, each slow output at the upper left corner of each scintillation block being multiplexed together, each slow output at the lower right corner of each scintillation block being multiplexed together and each slow output at the lower left corner of each scintillation block being multiplexed together.

According to another aspect of the invention, there is provided a method for distinguishing one scintillation event from a plurality of scintillation events at a series of scintillation blocks that are multiplexed together comprising:

providing two or more scintillation blocks multiplexed together in series, each scintillation block comprising a scintillation photomultiplier (SiPM) board having a plurality of SiPM pixels, each respective one SiPM pixel of the plurality of SiPM pixels arranged proximal to a respective one corner of the respective scintillation block, each SiPM pixel having a fast output and a slow output;
each fast output on a respective scintillation block being multiplexed together for reporting a scintillation event on the respective scintillation block;
each slow output at the respective one corner of a first scintillation block being multiplexed to a slow output at a corresponding corner of at least a second scintillation block for determining energy of a scintillation event and relative location on a scintillation block where the scintillation event occurred;

detecting one scintillation event at the multiplexed fast outputs on a respective one scintillation block, said respective one scintillation block reporting the one scintillation event fast output to a processor, said processor recording the one scintillation event fast output and applying a time stamp to the one scintillation event fast output;

the one scintillation event being measured by the multiplexed slow outputs at each corner of the series of scintillation blocks, said slow outputs each reporting the respective one scintillation event slow output and the measurement of the respective one scintillation event slow output to the processor, said processor applying a time stamp to each of the respective one scintillation event slow outputs measurements;

said processor comparing scintillation event fast output time stamps and respective one scintillation event slow outputs and assigning a scintillation event fast output and scintillation event slow outputs to one scintillation event, thereby mapping the one scintillation event to a specific location on a specific scintillation block.

As discussed herein and as will be apparent to one of skill in the art, mapping the scintillation events to specific locations on specific scintillation blocks is one step in the process of generating PET images. Accordingly, this method can also be considered a method of generating a PET image.

Specifically, as the annihilation events are determined, this information is used for PET imaging using means known in the art. According, this method can also be considered a method for PET imaging of a patient comprising distinguishing one scintillation event from a plurality of scintillation events at a series of scintillation blocks that are multiplexed together as described above. As individual scintillation events are distinguished as described above, it is possible to assemble a PET image of a body portion of a patient using means known in the art.

As discussed above, each respective one scintillation block of the series of scintillation blocks that are multiplexed together is positioned such that each respective one scintillation block cannot be in coincidence with any other respective one scintillation block, for example, any other respective one scintillation block within the series of scintillation blocks that are multiplexed.

The fast output may be put into a TDC circuit.

The slow output voltage signal may be put into a 40 MHz ADC system.

As discussed herein, in some embodiments, four slow output voltage signals are measured. However, as will be appreciated by one of skill in the art, this is not necessarily a requirement of the invention and any number of slow output voltage signals may be measured.

In some embodiments, each respective one of the four slow outputs is measured at a corner of the scintillation block. That is, as shown in the Figures, there are 4 outputs detected, one at each corner of the scintillation block. As discussed herein, other arrangements are possible within the invention.

In some embodiments, each corner slow output of a given scintillation block is multiplexed to the corresponding corner slow output at an adjacent scintillation block.

That is, for example each lower right corner of each of the scintillation blocks will be multiplexed together, each of the upper right corner outputs will be multiplexed together, each of the lower left corner outputs will be multiplexed together and each of the upper left corner outputs will be multiplexed together.

While the preferred embodiments of the invention have been described above, it will be recognized and understood that various modifications may be made therein, and the appended claims are intended to cover all such modifications which may fall within the spirit and scope of the invention.

1. Two or more scintillation blocks multiplexed together in series, each scintillation block comprising a scintillation photomultiplier (SiPM) board having a plurality of SiPM pixels, each respective one SiPM pixel of the plurality of SiPM pixels arranged proximal to a respective one corner of the respective scintillation block, each SiPM pixel having a fast output and a slow output;
each fast output on a respective scintillation block being multiplexed together for reporting a scintillation event on the respective scintillation block;
each slow output at the respective one corner of a first scintillation block being multiplexed to a slow output at a corresponding corner of at least a second scintillation block for determining energy of a scintillation event and relative location on a scintillation block where the scintillation event occurred.

2. The scintillation blocks according to claim 1 wherein each respective one scintillation block of the series of scintillation blocks that are multiplexed together is positioned such that each respective one scintillation block cannot be in coincidence with any other respective one scintillation block

3. The scintillation blocks according to claim 1 wherein the scintillation blocks are multiplexed to a Collimator Control Board.

4. The scintillation blocks according to claim 1 wherein there are more than 2 scintillation blocks multiplexed in series.

5. The scintillation blocks according to claim 1 wherein each scintillation block has more than 3 corners.

6. The scintillation blocks according to claim 1 comprising 4 scintillation blocks multiplexed in series, each scintillation block having 4 corners.

7. The scintillation blocks according to claim 1 wherein the fast outputs are put into TDC circuits.

8. The scintillation blocks according to claim 1 wherein the slow outputs are put into a 40 MHz ADC system.

9. The scintillation blocks according to claim 1 further comprising a third scintillation block and a fourth scintillation block, said scintillation blocks being arranged axially, each scintillation block having an upper right corner, an upper left corner, a lower right corner and a lower left corner, each slow output at the upper right corner of each scintillation block being multiplexed together, each slow output at the upper left corner of each scintillation block being multiplexed together, each slow output at the lower right corner of each scintillation block being multiplexed together and each slow output at the lower left corner of each scintillation block being multiplexed together.

10. A method for distinguishing one scintillation event from a plurality of scintillation events at a series of scintillation blocks that are multiplexed together comprising:
providing two or more scintillation blocks multiplexed together in series, each scintillation block comprising a scintillation photomultiplier (SiPM) board having a plurality of SiPM pixels, each respective one SiPM pixel of the plurality of SiPM pixels arranged proximal to a respective one corner of the respective scintillation block, each SiPM pixel having a fast output and a slow output;
each fast output on a respective scintillation block being multiplexed together for reporting a scintillation event on the respective scintillation block;
each slow output at the respective one corner of a first scintillation block being multiplexed to a slow output at a corresponding corner of at least a second scintillation block for determining energy of a scintillation event and relative location on a scintillation block where the scintillation event occurred;
detecting one scintillation event at the multiplexed fast outputs on a respective one scintillation block, said respective one scintillation block reporting the one scintillation event fast output to a processor, said processor recording the one scintillation event fast output and applying a time stamp to the one scintillation event fast output;
the one scintillation event being measured by the multiplexed slow outputs at each corner of the series of scintillation blocks, said slow outputs each reporting the respective one scintillation event slow output and the measurement of the respective one scintillation event slow output to the processor, said processor applying a time stamp to each of the respective one scintillation event slow outputs measurements;
said processor comparing scintillation event fast output time stamps and respective one scintillation event slow outputs and assigning a scintillation event fast output and scintillation event slow outputs to one scintillation event, thereby mapping the one scintillation event to a specific location on a specific scintillation block.

11. The method according to claim 10 wherein each respective one scintillation block of the series of scintillation blocks that are multiplexed together is positioned such that each respective one scintillation block cannot be in coincidence with any other respective one scintillation block.

12. The method according to claim 10 wherein the scintillation blocks are multiplexed to a Collimator Control Board.

13. The method according to claim 10 wherein there are more than 2 scintillation blocks multiplexed in series.

14. The method according to claim 10 wherein each scintillation block has more than 3 corners.

15. The method according to claim 10 comprising 4 scintillation blocks multiplexed in series, each scintillation block having 4 corners.

16. The method according to claim 10 wherein the fast outputs are put into TDC circuits.

17. The method according to claim 10 wherein the slow outputs are put into a 40 MHz ADC system.

18. The method according to claim 10 further comprising a third scintillation block and a fourth scintillation block, said scintillation blocks being arranged axially, each scintillation block having an upper right corner, an upper left corner, a lower right corner and a lower left corner, each slow output at the upper right corner of each scintillation block being multiplexed together, each slow output at the upper left corner of each scintillation block being multiplexed together, each slow output at the lower right corner of each scintillation block being multiplexed together and each slow output at the lower left corner of each scintillation block being multiplexed together.
